(19) United States
(12) Patent Application Publication
(10) Pub. No.: US 2026/0034305 A1
(43) Pub. Date: Feb. 5, 2026

(54) INJECTION DEVICE

(71) Applicant: Owen Mumford Limited, Woodstock, Oxfordshire (GB)

(72) Inventors: Robert William BRUNS, Woodstock, Oxfordshire (GB); Dheeraj RAMAKRISHNAN, Woodstock, Oxfordshire (GB)

(21) Appl. No.: 19/121,535

(22) PCT Filed: Oct. 17, 2023
(86) PCT No.: PCT/EP2023/078888

§ 371 Date: Apr. 16, 2025

(30) Foreign Application Priority Data
Oct. 17, 2022 (GB) 2215312.6

(51) Int. Cl. A61M 5/28; A61M 5/20; A61M 5/315; A61M 5/32

(52) U.S. Cl. CPC A61M 5/281; A61M 5/31568; A61M 5/3243; A61M 2005/208; A61M 2205/3592; A61M 2205/50; A61M 2205/8212

(57) ABSTRACT

An injection device comprising a main housing, a drive mechanism for providing motive force to deliver medication, and a shield attached to a proximal end of the main housing, the shield being coupled to the drive mechanism so that inward movement of the shield causes release of the drive mechanism to provide said motive force, and the shield being biased towards an extended position. The injection device further comprises a plurality of sensors including an in-use sensor coupled between the main housing and the shield, and a monitoring and reporting unit electrically coupled to the sensors for monitoring use. In a lower power mode, the in-use sensor is monitored whilst the other sensors and components are inoperable and, in a higher power mode, the other sensors and components are operable. Upon detection by the in-use sensor of inward movement of the shield the monitoring and reporting unit is switched from the lower power mode to the higher power mode.

### TECHNICAL FIELD

The present invention relates to injection devices for drug delivery and provided with electronic monitoring operable in a low power mode.

### BACKGROUND

Injection devices are commonly used to deliver many different types of drug. In the majority of injection devices, the force to deliver an injection is provided by powerful helical springs, either compressed and released to provide the delivery force or expanded and contracted to provide that force. In some cases, the same or a different spring mechanism provides a needle insertion force to cause a syringe or cartridge needle tip to penetrate a user's skin, prior to drug delivery.

It is of course of the utmost importance that medication delivery regimens are followed precisely, e.g. in terms of drug delivered per injection and the timing of injections. This has traditionally relied on careful use by patients or their medical practitioners or carers. Particularly for self-injections, where users may be elderly or otherwise frail, it can be very difficult to follow a set regimen and injections may be missed or too many carried out, or the wrong dose delivered, potentially leading to serious consequences.

With the widespread use of smartphones and other wirelessly connected personal devices, interest is growing in the use of autoinjectors provided with electronic monitoring means and wireless connectivity, such as will allow the automatic monitoring of injections and reporting to user devices, possibly with further onward reporting to some central monitoring service or medical practice. Whilst such electronic monitoring and reporting functionality may be used in conjunction with electrical/electronic drive means for injection delivery (and needle insertion), at least for the time being it is considered preferable to continue using a mechanical drug delivery mechanism to ensure reliability and the possibility to perform an injection should electrical power (e.g. from a rechargeable battery) be unavailable.

WO2019086718 describes an implementation of a mechanically powered autoinjector with electronic monitoring. Detection relies upon the use of micro-switches although it is suggested that other means for detection may be used.

Whilst relatively low power monitoring and wireless reporting functions can be implemented within an injection device, power consumption continues to be a challenge, particularly where devices are expected to have a life of several years without the need to recharge or replace batteries; again ease of operation is critical for elderly and frail users.

### SUMMARY

According to a first aspect of the present invention there is provided an injection device for delivering a dose of medication into a patient from a medication containing syringe or cartridge having a needle affixed thereto. The injection device comprises a main housing for receiving the syringe or cartridge, a drive mechanism located substantially within the main housing for providing motive force to deliver medication into the patient from the syringe or cartridge, and a shield defining an opening therethrough, attached to a proximal end of the main housing and movable relative to the housing between an extended position in which a needle tip is substantially shielded and a retracted position in which the needle tip extends through the opening in the shield, the shield being coupled to the drive mechanism so that movement of the shield from the extended position to the retracted position causes or permits a release of the drive mechanism to provide said motive force, and the shield being biased towards the extended position. The injection device further comprises a plurality of sensors including an in-use sensor, the in-use sensor coupled between the main housing and the shield to detect movement of the shield between the extended position and the retracted position, and an electrically powered monitoring and reporting unit contained substantially within the main housing and electrically coupled to the plurality of sensors for monitoring use of the injection device including successful medication delivery and for reporting said use to an external device, via a wireless interface of the unit, the unit being operable in at least one of a lower power mode and a higher power mode. In the lower power mode, at least the in-use sensor is monitored by the unit whilst the other sensors and components are inoperable or otherwise do not consume power and, in the higher power mode, the other sensors and components are operable, and wherein detection, by the in-use sensor, of movement of the shield from the extended position to the retracted position causes the monitoring and reporting unit to switch from the lower power mode to the higher power mode.

The housing may comprise first and second main housing parts movable relative to one another between an open position in which the syringe or cartridge can be inserted into a receiving slot defined within the first main housing part and a closed position in which the inserted syringe or cartridge is securely retained in the receiving slot and within the housing, wherein the shield comprises a first shield part coupled to the first main housing part and a second shield part coupled to the second main housing part such that the shield parts come together when the main housing parts are in the closed position to form a substantially unitary shield. The shield is further coupled to the drive mechanism by the first shield part and the first main housing part, whilst the in-use sensor is coupled between the second shield part and the second main housing part.

Detection, by the in-use sensor, of movement of the shield from the retracted position to the extended position may cause the monitoring and reporting unit to switch from the higher power mode to the lower power mode.

The monitoring and reporting unit may comprise a processor and, in the lower power and higher power modes, the processor is configured to detect interrupt signals at an input or inputs coupled to the in-use sensor.

The in-use sensor may comprise a micro-switch. Said other sensors may include at least a sensor to detect commencement of medication delivery and a sensor to detect end of medication delivery.

According to a second aspect of the present invention there is provided an injection device for delivering a dose of medication into a patient from a medication containing syringe or cartridge having a needle affixed thereto. The injection device comprises a main housing for receiving the syringe or cartridge, a drive mechanism located substantially within the main housing for providing motive force to deliver medication into the patient from the syringe or cartridge, a trigger coupled to the drive mechanism so that movement of the trigger from a first position to a second position relative to the main housing causes or permits a release of the drive mechanism to provide said motive force, and a plurality of sensors including an in-use sensor, the in-use sensor coupled between the main housing and the trigger to detect movement of the trigger from the first to the second position. The injection device further comprises an electrically powered monitoring and reporting unit contained substantially within the main housing and electrically coupled to the plurality of sensors for monitoring use of the injection device including successful medication delivery and for reporting said use to an external device, via a wireless interface of the unit, the unit being operable in at least one of a lower power mode and a higher power mode. In the lower power mode, at least the trigger is monitored by the unit whilst the other sensors and components are inoperable or otherwise do not consume power and, in the higher power mode, the other sensors and components are operable, and wherein detection, by the in-use sensor, of movement of the trigger from the first to the second position causes the monitoring and reporting unit to switch from the lower power mode to the higher power mode.

### BRIEF DESCRIPTION OF THE DRAWINGS

FIG. 1A-C show an autoinjector in a (A) closed, (B) partially open and (C) open state;

FIG. 2 shows a safety syringe;

FIG. 3 shows a capped end of the autoinjector of FIGS. 1A-C;

FIGS. 4A and 4B show partial cross-sections of the autoinjector of FIGS. 1A-C; and

FIG. 5 shows features of the autoinjector of FIGS. 1 to 4 within the detail A of FIG. 1C.

### DETAILED DESCRIPTION

As noted above it is desirable to minimise the power consumption of injection devices making use of electronic components including sensors, for example to extend the life of a non-rechargeable device or to maximise the intervals between charging in the case of a rechargeable device. This may be achieved, at least in part, by providing a sensor or sensors for detecting an injection initiation step, this detection being used to change the power operating mode of the device from a low power or “sleep” mode to a higher power mode in which further sensors and/or monitoring functions are switched on.

The terms “forward” or “front” are used here to refer to the needle side or injection site end of the autoinjector, whereas the term “rear” refers to the end of the autoinjector remote from the needle or injection site.

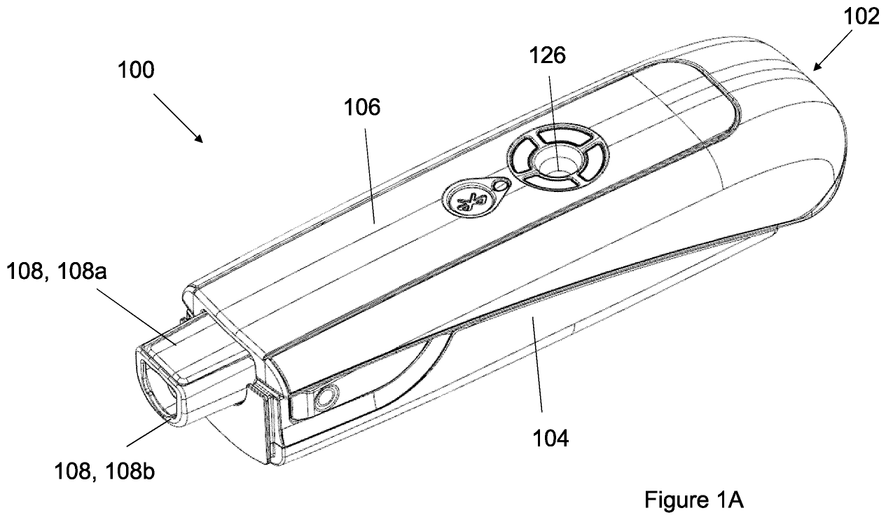

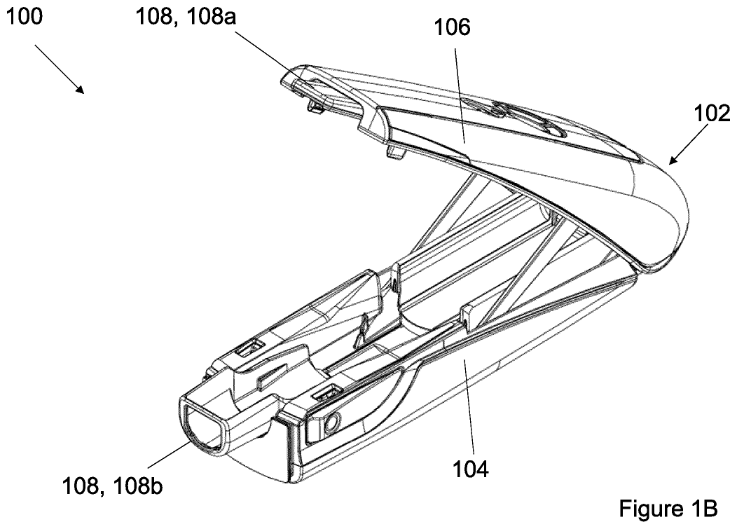

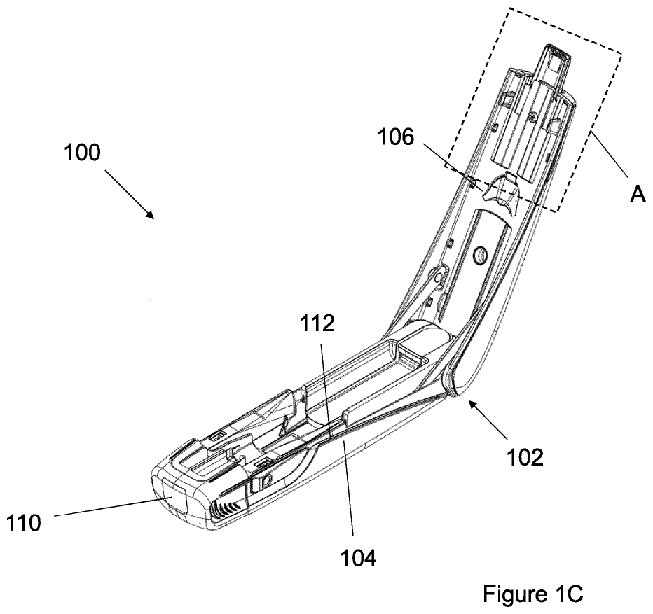

FIGS. 1A-C show an embodiment of an autoinjector 100 in: A) a closed state; B) a partially open state; and C) a fully open state.

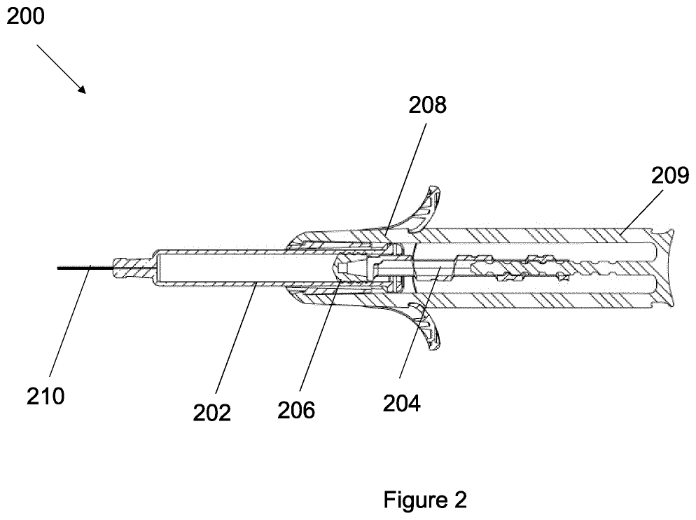

The autoinjector 100 comprises a housing 102 which includes a main body 104 and a lid 106 that are hingedly connected so as to permit opening and closing of the housing. The autoinjector further comprises a plurality of component parts contained within the housing. A syringe such as the syringe 200 of FIG. 2 (not shown in FIGS. 1A-C) is receivable within the housing in a slot 112 defined in the main body. The lid 106 of the autoinjector 100 includes a through-hole 126 positioned so that a surface of a plunger driver 116, the operation of which is described below, is viewable once firing is complete. That surface is vividly coloured in contrast to other parts visible through the through-hole prior to and during drug delivery to thereby provide a visual indication to the user that drug delivery is complete.

As shown most clearly in FIGS. 1A and 1B, the autoinjector 100 further comprises a shroud 108, formed from a lower part 108a and an upper part 108b. The lower and upper parts are respectively coupled to the main body 104 and lid 106 so that the parts 108a, 108b separate as the housing 102 is opened to allow insertion of a syringe 200 and come together to form the unitary shroud 108 when the housing is closed. The shroud 108 defines an aperture through which the needle 210 of a syringe 200 at least partially extends, when a syringe is received in the autoinjector 100. The lower part and upper part 108a, 108b comprise slidable connections with the main body 104 and lid 106 respectively, to permit movement between an extended position where the end of the syringe needle is substantially covered by the shroud, and a retracted position where the end of the syringe needle is exposed. The shroud parts are separately biased towards the extended position so that the needle of a syringe in the autoinjector remains substantially covered prior to an injection.

As shown in FIG. 1C, the autoinjector 100 includes a removable cap 110 which is normally in place prior to an injection being performed. For ease of understanding, the cap is omitted from FIGS. 1A and 1B. In the configuration shown, the cap fits slidably over the lower shroud part 108b and further abuts against a front end of the main body 104. The cap 110 prevents user access to the shroud and hence accidental firing while the cap is in place.

FIG. 2 illustrates a safety syringe 200 suitable for use with the described autoinjector 100. Such a syringe 200 is described in detail in WO2019086718. It is sufficient here to note that the syringe comprises a syringe body 202 for containing a medicament, a syringe plunger 204 that engages with a bung 206 within the syringe body, a needle shield 208 coupled to a safety plunger 209, and a needle 210. The coupling between the syringe plunger and the safety plunger/needle shield is such that the needle shield 208 is deployed around the needle of the syringe so as to substantially cover the needle following delivery of the medicament from the syringe body. This coupling is described in detail in WO2019/086718.

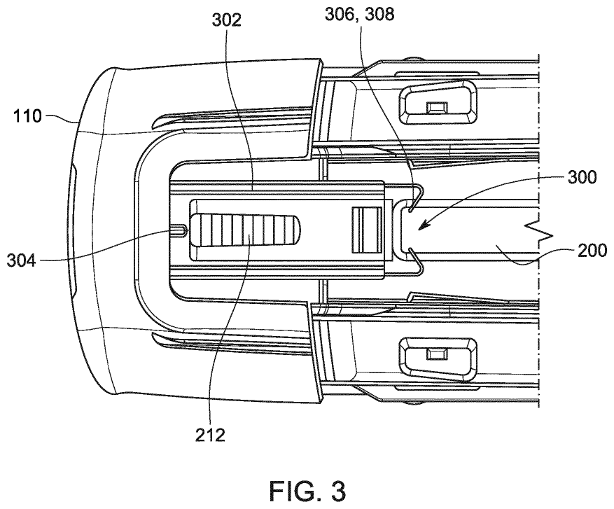

In general, syringes, including safety syringes, are routinely provided with a protective rigid needle shield (RNS) which require removal before a syringe can be used (the RNS is not shown in FIG. 2). To this end, the cap 110 also operates, in a known way, as an RNS remover 300. FIG. 3 shows a top plan view of the end of the autoinjector with the cap in place. The autoinjector 100 is in the open state such that the RNS remover and the end of a syringe 200 with an attached RNS 212 are visible. The RNS remover comprises a side wall 302 extending away from the cap, which defines a passageway 304 for receiving the RNS when the cap is fitted to the autoinjector. The front end of the side wall terminates with a gripping member 306. The gripping member is configured to allow easy insertion of the RNS 212 into the passageway whilst preventing its withdrawal thereafter. The RNS can therefore be removed from the syringe as the cap is removed.

In the configuration shown in FIG. 3, the gripping member 306 comprises projections 308 which extend inwardly into the passageway 304, angled away from the front end the side wall 302. As the syringe 200 with RNS is inserted into the slot 112 of the main body 104, the projections 308 are able to flex outwardly, whereas any return motion is prevented by the projections 308 as they come into engagement with the RNS 212.

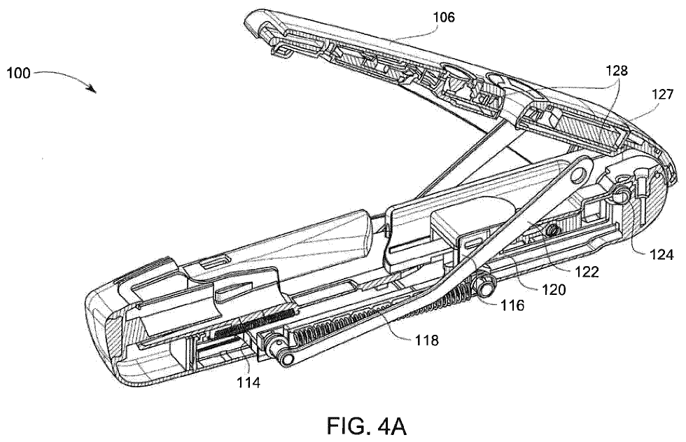

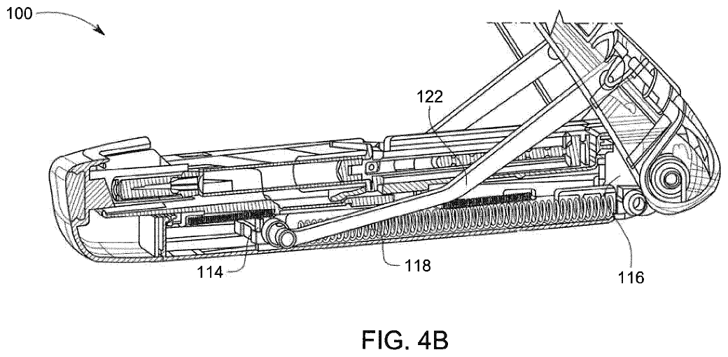

FIGS. 4A and 4B show partial cross-sectional views of the autoinjector 100 during various stages of priming to illustrate the presence and operation of further internal components during lid 106 opening and closing strokes. In particular, it can be seen that the autoinjector 100 comprises a shuttle 114 which is operable to move between a first forward position and a second rearward position along a shuttle guide 120 on the main body 104 of the housing 102, a plunger driver 116 for driving the syringe plunger 204, and a biasing element 118 which couples the shuttle and plunger driver 116. The shuttle and plunger driver are slidably connected to the shuttle guide 120 to permit rearward and forward movement within the housing 102. Unlike the plunger driver, the shuttle is also fixedly connected to the lid 106 via two arm members 122. The plunger driver comprises a rearmost member 117, referred to here as a “push member”, that is located in use behind the rear end of the plunger of an inserted syringe.

The biasing element 118 comprises two extension springs on either side of the device, although only one is visible in the drawings. Prior to any priming, these springs are under slight tension so as to hold the plunger driver 116 and shuttle 114 together. It should therefore be noted that priming, in the context of the extension springs, refers to the process of further tensioning the extension springs into a state whereupon firing can be initiated.

Each of the shuttle guide 120 and the plunger driver 116 comprise part of a latching arrangement, which are configured to cooperate to secure the plunger driver at the rear end of the autoinjector 100. A suitable latching arrangement is described in WO2022179832.

The autoinjector 100 further comprises a torsion spring 124 arranged at the hinged connection between the lid 106 and main body 104 of the autoinjector 100. The torsion spring is coupled to both the lid and main body. In the embodiment shown, one end of the torsion spring is attached to the lid, and the opposing end is attached to the main body of the autoinjector.

Priming of the autoinjector on the lid opening stroke (FIG. 4A) and the lid closing stroke (FIG. 4B) is now described. WO2021058474 describes operation of a similar autoinjector, except that the biasing element described therein further includes a compression spring.

As the lid 106 is opened, the arm members 122 which couple the lid and shuttle 114 together cause the shuttle to move rearwards from the first position to the second position. The shuttle is in constant engagement with the plunger driver 116 so that its rearward travel causes the same rearward travel for the plunger driver. The extension springs coupled between them therefore remains un-primed (i.e. further extended) during lid opening. Near the end of lid opening stroke, the latching arrangement part on the shuttle guide 120 and the plunger driver are brought together such that they are able to cooperate to secure the plunger driver at the rear end of the autoinjector 100.

Lid 106 opening also causes the end of the torsion spring 124 attached to the lid to rotate about its spring axis relative to the opposing end of the torsion spring. This primes the torsion spring on lid opening. When primed, the torsion spring produces a restoring force which tends to urge the lid closed.

Upon closing of the lid 106, whilst the shuttle 114 is free to move forwards along the shuttle guide 120 to the first position, the plunger driver 116 is held at the rear of the autoinjector by the latching arrangement. Thus, during the lid closing stroke, the shuttle and plunger driver separate and the extension springs coupled between them are primed (i.e., further tensioned).

As has already been noted above, the primed torsion spring 124 urges the lid 106 closed. This assists in priming the extension springs 118 during closing, whilst requiring a minimal force to prime the torsion spring during opening. This is important for users of autoinjectors who would otherwise find it difficult to apply the necessary force to close the lid.

Firing of the autoinjector is now described. The firing mechanism is described in more detail in WO2022179832.

To fire the loaded and primed autoinjector, the user urges the front end of the autoinjector 100 into contact with an injection site (e.g., a user's skin). This causes the shroud parts 108a, 108b to move into the retracted position against their biases (e.g., respective springs). As the shroud retracts into the housing 102, the lower shroud 108b permits or causes release of the latching arrangement and the primed extension springs 118a, 118b. The restoring force of the extension springs, acting on the plunger driver 116, drives the plunger driver, and specifically the push member 117, forwards to depress the syringe plunger and force the drug from out of the syringe needle into the injection site.

Referring now to FIG. 4A, this illustrates an electronics module 127 located within the lid 106 of the housing 102. Components within the housing, including for example a processor and memory, operate as a computer, whilst other components may operate as a radio transceiver for sending and receiving data to an external device such as a smartphone. The transceiver may utilise a wireless interface such as a Bluetooth™ or WiFi interface. The radio transceiver might alternatively be configured to communicate with some remote service via, for example, a cellular (mobile) radio network. The module 127 is located on and electrically connected to a printed circuit board (PCB) 128. The autoinjector further comprises a plurality of sensors configured to monitor or detect respective operating steps and/or processes of the autoinjector. These sensors may be attached to the PCB or otherwise electrically coupled thereto, facilitating electrical communication between the sensors and the electronics module. Although not shown in the Figures the autoinjector comprises an electrical energy source, typically a battery.

This may be contained within the electronics module 127.

Examples of sensors (not shown in the Figures) include sensors for:
detecting commencement of movement of the plunger driver from its rearmost position signalling start of drug delivery;
detecting end of movement of the plunger driver signalling end of drug delivery detecting opening and/or closing of the lid; and/or
detecting successful insertion of a syringe into the housing.

Sensors of the type identified above will typically consume energy whilst in an active monitoring state, even if the device is not about to be used. An obvious solution to this problem is to provide an on/off switch that a user operates when an injection is about to be made and after an injection has been completed. This however relies on a user to correctly operate the switch, particularly after completing an injection. It may also assume that all switches can be on and off in synchronisation.

An improved solution is to provide a dedicated sensor, referred to her as an “in-use” sensor, that is configured to detect commencement of an actual injection, i.e. needle insertion into a patient's skin and, optionally, needle removal, the output signal of the in-use sensor being used to switch the device from a low power or sleep mode to a relatively high power mode. In the low power mode, only the in-use sensor is supplied with power or otherwise monitored, whilst other sensors of the device are switched off and/or not monitored. In some cases, in the low power mode, a limited number of other sensors may be on and/or monitored, e.g. sensors for detecting opening and closing of the lid. In the higher power mode, all sensors of the device are on and/or monitored (or at least those other sensors required during an injection phase).

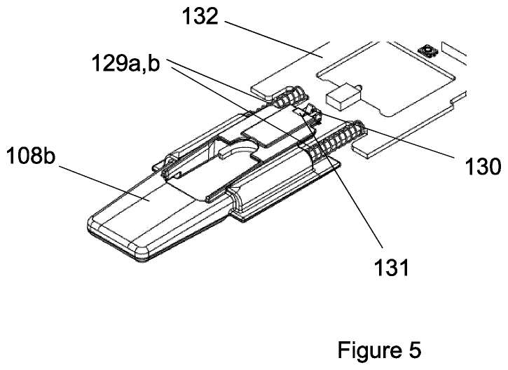

FIG. 5 illustrates features of a front end of the injection device, within a region indicated generally by the broken line A in FIG. 1C. Shown in particular are the upper part 108b of the shroud 108 and an internal front end 132 of the lid 106. Also shown are a pair of springs 129a,b coupled between the upper part 108b and the lid which act to bias the upper part in a forward direction as discussed above. When depressed, the upper part 108b slides across a front end 132 of the lid part that it engages, against the applied bias of the springs 129a,b. An in-use sensor 130 in the form of a micro-switch is located on the front end 132 of the lid 106. The in-use sensor is positioned such that it is triggered as a feature 131 at the rear end of the upper part engages the switch. This event occurs at some point on the inward travel of the upper part, as the shroud is depressed by the reaction force when the shroud 108 is pressed against the user's skin. This is typically at or close to the point when the needle tip penetrates the user's skin.

The in-use sensor 130 is electrically connected to the electronics module 127, e.g. via the PCB 128. The module may apply a small voltage across a switch of the sensor, with closure of the switch resulting in a voltage signal being provided to an interrupt line of the processor within the electronics module. This operation allows the processor to detect depressing and subsequent release of the shroud 108 and in turn switch its mode of operation between the above mentioned low power and relatively higher power modes. It will be appreciated that the autoinjector may operate in the higher power mode for only relatively short periods of time, e.g. i.e. the time taken to perform an injection. This period might be a few seconds or a few tens of seconds. This allows for a very significant reduction in the power consumed by the device in turn greatly extending the life of the power source (e.g. battery) and possibly of the autoinjector in the case where the power source is not rechargeable or replaceable,

The skilled reader will be able to envisage further embodiments of the invention without departing from the scope of the appended claims. It will be appreciated for example that, rather than being an electro-mechanical micro-switch, the in-use sensor may be an optical sensor or other type of proximity sensor. Alternative, or additional means for switching the device from a low power mode to a high power mode may also be provided. For example, where the device is provided with Bluetooth™ functionality for communicating sensed and other data to an external computer system, e.g. a user's smartphone, operation of a Bluetooth switch may cause this mode switch. As an alternative to integrating the switch into the shroud, it may be integrated into a trigger button, where depression of the trigger button initiates an injection sequence. Such manually operated trigger buttons are well known in the art.

Further, although the present invention has been described in relation to an autoinjector the skilled person will also appreciate that it could be applied to other injection devices such as a pen device where a user selects the amount of medicament to be dispensed from the cartridge or syringe. Further the in-use sensor may be a firing button or other trigger used to begin the injection sequence.

1. An injection device for delivering a dose of medication into a patient from a medication containing syringe or cartridge having a needle affixed thereto, the injection device comprising:
a main housing for receiving the syringe or cartridge;
a drive mechanism located substantially within the main housing for providing motive force to deliver medication into the patient from the syringe or cartridge;
a shield defining an opening therethrough, attached to a proximal end of the main housing and movable relative to the housing between an extended position in which a needle tip is substantially shielded and a retracted position in which the needle tip extends through the opening in the shield, the shield being coupled to the drive mechanism so that movement of the shield from the extended position to the retracted position causes or permits a release of the drive mechanism to provide said motive force, and the shield being biased towards the extended position;
a plurality of sensors including an in-use sensor, the in-use sensor coupled between the main housing and the shield to detect movement of the shield between the extended position and the retracted position; and
an electrically powered monitoring and reporting unit contained substantially within the main housing and electrically coupled to the plurality of sensors for monitoring use of the injection device including successful medication delivery and for reporting said use to an external device, via a wireless interface of the unit, the unit being operable in at least one of a lower power mode and a higher power mode,
wherein, in the lower power mode, at least the in-use sensor is monitored by the unit whilst the other sensors and components are inoperable or otherwise do not consume power and, in the higher power mode, the other sensors and components are operable, and wherein detection, by the in-use sensor, of movement of the shield from the extended position to the retracted position causes the monitoring and reporting unit to switch from the lower power mode to the higher power mode.

2. An injection device according to claim 1, the housing comprising first and second main housing parts movable relative to one another between an open position in which the syringe or cartridge can be inserted into a receiving slot defined within the first main housing part and a closed position in which the inserted syringe or cartridge is securely retained in the receiving slot and within the housing,
wherein the shield comprises a first shield part coupled to the first main housing part and a second shield part coupled to the second main housing part such that the shield parts come together when the main housing parts are in the closed position to form a substantially unitary shield,
the shield being coupled to the drive mechanism by the first shield part and the first main housing part, and
the in-use sensor being coupled between the second shield part and the second main housing part.

3. An injection device according to claim 1 wherein detection, by the in-use sensor, of movement of the shield from the retracted position to the extended position causes the monitoring and reporting unit to switch from the higher power mode to the lower power mode.

4. An injection device according to claim 1 wherein the monitoring and reporting unit comprises a processor and, in the lower power and higher power modes, the processor is configured to detect interrupt signals at an input or inputs coupled to the in-use sensor.

5. An injection device according to claim 1, wherein the in-use sensor comprises a mechanical switch.

6. An injection device according to claim 1, wherein the in-use sensor comprises a micro-switch.

7. An injection device according to claim 1, wherein said other sensors include at least a sensor to detect commencement of medication delivery and a sensor to detect end of medication delivery.

8. An injection device for delivering a dose of medication into a patient from a medication containing syringe or cartridge having a needle affixed thereto, the injection device comprising:
a main housing for receiving the syringe or cartridge;
a drive mechanism located substantially within the main housing for providing motive force to deliver medication into the patient from the syringe or cartridge;
a trigger coupled to the drive mechanism so that movement of the trigger from a first position to a second position relative to the main housing causes or permits a release of the drive mechanism to provide said motive force;
a plurality of sensors including an in-use sensor, the in-use sensor coupled between the main housing and the trigger to detect movement of the trigger from the first to the second position; and
an electrically powered monitoring and reporting unit contained substantially within the main housing and electrically coupled to the plurality of sensors for monitoring use of the injection device including successful medication delivery and for reporting said use to an external device, via a wireless interface of the unit, the unit being operable in at least one of a lower power mode and a higher power mode,
wherein, in the lower power mode, at least the trigger is monitored by the unit whilst the other sensors and components are inoperable or otherwise do not consume power and, in the higher power mode, the other sensors and components are operable, and wherein detection, by the in-use sensor, of movement of the trigger from the first to the second position causes the monitoring and reporting unit to switch from the lower power mode to the higher power mode.

9. An injection device according to claim 8, the housing comprising first and second main housing parts movable relative to one another between an open position in which the syringe or cartridge can be inserted into a receiving slot defined within the first main housing part and a closed position in which the inserted syringe or cartridge is securely retained in the receiving slot and within the housing,
wherein the shield comprises a first shield part coupled to the first main housing part and a second shield part coupled to the second main housing part such that the shield parts come together when the main housing parts are in the closed position to form a substantially unitary shield,
the shield being coupled to the drive mechanism by the first shield part and the first main housing part, and
the in-use sensor being coupled between the second shield part and the second main housing part.

10. An injection device according to claim 8 wherein detection, by the in-use sensor, of movement of the shield from the retracted position to the extended position causes the monitoring and reporting unit to switch from the higher power mode to the lower power mode.

11. An injection device according to claim 8 wherein the monitoring and reporting unit comprises a processor and, in the lower power and higher power modes, the processor is configured to detect interrupt signals at an input or inputs coupled to the in-use sensor.

12. An injection device according to claim 8, wherein the in-use sensor comprises a micro-switch.

13. An injection device according to claim 8, wherein said other sensors include at least a sensor to detect commencement of medication delivery and a sensor to detect end of medication delivery.
